(19) United States
(12) Patent Application Publication
(10) Pub. No.: US 2022/0202841 A1
(43) Pub. Date: Jun. 30, 2022

(54) DERMATOLOGICAL FORMULATIONS FOR TREATMENT OF DERMATITIS

(71) Applicant: Erine A. Kupetsky, Allentown, NJ (US)

(72) Inventor: Erine A. Kupetsky, Allentown, NJ (US)

(21) Appl. No.: 17/559,187

(22) Filed: Dec. 22, 2021

Related U.S. Application Data
(60) Provisional application No. 63/199,447, filed on Dec. 29, 2020.
(60) Provisional application No. 63/199,422, filed on Dec. 24, 2020.

(51) Int. Cl. A61K 31/7048; A61K 9/00; A61K 31/4164

(52) U.S. Cl. CPC A61K 31/7048; A61K 31/4164; A61K 9/0014

(57) ABSTRACT

Pharmaceutical formulations containing an avermectin compound, a nitroimidazole compound, and a macrolide antibiotics compound in a suitable topical delivery system; and processes for preparing the formulations; and methods of using the formulations for the treatment of dermatological conditions, or disorders, in particular peri-oral dermatitis.

### CROSS-REFERENCE TO RELATED APPLICATION

The present application claims the benefit of priority under 35 U.S.C. § 119(e) of U.S. Provisional Application Ser. No. 63/199,447, filed Dec. 29, 2020 and 63/199,422, filed Dec. 24, 2020. The entire disclosures of the applications noted above are incorporated herein by reference.

### BACKGROUND OF THE INVENTION

### Technical Field of the Invention

The present invention relates to pharmaceutical Formulations containing a) an avermectin compound or a pharmaceutically acceptable salt thereof; b) a nitroimidazole compound or a pharmaceutically acceptable salt thereof, c) a macrolide antibiotics compound in a suitable topical delivery system. This invention also relates to a process for preparing such pharmaceutical Formulations and using such pharmaceutical Formulations for the treatment of dermatological conditions, or disorders, in particular peri-oral dermatitis.

### BACKGROUND AND RELATED ART

Dermatological conditions are often associated with increased sensitivity of the skin caused by an inflammatory process that affects the face and can further be manifested by reddening of the face, hot flashes, and facial erythema. Perioral dermatitis (POD) is particularly a clinically distinct reaction pattern of the human skin which predominatly effects young women. Overuse of cosmetic products, and intermittent use of glucocortiosteroids are frequently associated with the occurrence and development of this condition. Subjective complaints may include burning sensations, tense feeling of the skin, but not itching, while the objective symptoms frequently include erythema, papulation, papulopustules, papulovesicules and scaling.

There is currently no standard approach for treatment of this condition. Instead, the treatment is generally guided based on physician's individual experience and use of topical antibiotics, antiacne, antifungal and even immunosuppressive agents. Lack of understanding of the ethiology of this condition further limits the range of therapeutic options for the treatment of this condition. As such, there remains a need in the art to provide effective treatment plans for patients suffering from this condition.

### SUMMARY OF THE INVENTION

The present invention addresses the need in the art. The present application relates to dermal formulations, cream, ointment, gel, including foamable and foam formulations comprising a) an avermectin compound or a pharmaceutically acceptable salt thereof; b) a nitroimidazole compound or a pharmaceutically acceptable salt thereof, and c) a macrolide antibiotics compound or a pharmaceutically acceptable salt thereof which are stable for the extent of their therapeutic use.

An aspect of the present invention is a formulation containing a) an avermectin compound or a pharmaceutically acceptable salt thereof in amounts ranging from about 0.001% to about 35% by weight; b) a nitroimidazole compound or a pharmaceutically acceptable salt thereof in amounts ranging from about 0.1% to about 25% by weight, and c) a macrolide antibiotics compound or a pharmaceutically acceptable salt thereof in amounts of about 0.1% to about 30% by weight and a topical carrier that includes an organic or an aqueous solvent, a thickening agent, a surfactant, an oil moiety, a buffering agent, a pH adjusting agent, or any other suitable excipient to facilate and enhance the intradermal penetration of the active ingredients.

In at least some embodiments, the topical carrier contains a surfactant that is selected from anionic, cationic, nonionic, amphoteric surfactants and a mixture thereof. In other embodiments, the carrier can include a fatty substances selected from the group consisting of fatty acids, fatty alcohols, waxes, gums, and mixtures thereof.

In certain embodiments, the carrier may include from 0.1 to 50% by weight of suitable solvents. In certain embodiments, the solvent may be polar, non-polar and a mixture of polar and non-polar solvents. In certain embodiments, the solvents may include water, C1-C20 alcohol, propylene glycol, N-methyl-2-pyrrolidone, dimethylsulfoxyde, polysorbate 80, poloxamer 124, phenoxyethanol, oleyl alcohol, isostearic acid, diisopropyl adipate, polypropylene glycol-15 stearyl ether (“PPG-15 stearyl ether”), octyl dodecanol, ethyl oleate, C12-C15 alkyl benzoate and mixtures thereof.

Other embodiments of the present invention may include a carrier that contains surfactants such as an ionic or non-ionic surfactants. In some embodiments, the formulation of present invention may have a viscosity within a range of from about 1,000 centipoise to about 100,000 centipoise at 25° C.

In some embodiments, the solvent is an aqueous solvent such as water, in an amount of about 1% to about 95% by weight of the Formulation; a buffering agent in an amount of about 1% to about 10% by weight of the formulation; and a surfactant in an amount of about 0.5% to about 25% by weight of the Formulation.

In some embodiment, the avermectin compound may be any one of ivermectin, invermectin, avermectin, abamectin, doramectin, eprinomectin, selamectin and a combination thereof. In a particular preferred embodiment, the avermectin compound is ivermectin. In some embodiments, the macrolide compound may be any one of erythromycin, clarithromycin, azithromycin, spiramycin, fidaxomicin and any combinations thereof. In other embodiments, the nitroimidazole compound is metronidazole.

In other embodiments, the formulations of the present invention contains a macrolide compound that is selected from the group erythromycin, clarithromycin, azithromycin, spiramycin, fidaxomicin and any combination thereof. In some embodiments, the macrolide is erythromycin or a pharmaceutically suitable salt or ester thereof.

In some embodiments, the formulation may contain an additional active agent selected from an active herbal extract, an acaricides, an age spot and keratose removing agent, an allergen, an alpha hydroxyl acid, an analgesic agent, an androgen, an antiacne agent, an antiallergic agent, an antiaging agent, an antibacterial agent, an antibiotic, an antiburn agent, an anticancer agent, an antidandruff agent, an antidepressant, an antidermatitis agent, an antiedemic anent, an antifungal agent, an antihistamine, an antihelminth agent, an anti-hyperkeratosis agent, an anti-infective agent, an antiinflarnmatory agent, an antiirritant, an antilipemic agent, an antimicrobial agent, an antimycotic agent, an antioxidant, an antiparasitic agent, an antiproliferative agent, an antipruritic agent, an antipsoriatic agent, an antirosacea agent, an antiseborrheic agent; an antiseptic agent, an antiswelling agent, an antiviral agent, an anti-wart agent, an anti-wrinkle agent, an antiyeast agents, an astringent, a beta-hydroxy acid, benzoyl peroxide, a topical cardiovascular agent, a chemotherapeutic agent, a corticosteroid, an immunogenic substance, a dicarboxylic acid, a disinfectant, an estrogen, a fungicide, a hair growth regulator, a haptene, a hormone, a hydroxy acid, an immunosuppressant, an immunoregulating agent, an immunomodula or, an immunostimulant, an insecticide, an insect repellent, a keratolytic agent, a lactam, a local anesthetic agent, a lubricating agent, a masking agent, a metal, a metal oxide, a mitocide, a neuropeptide, a non-steroidal anti-inflammatory agent, an oxidizing agent, a pediculicide, a peptide, a pesticide, a protein, a photodynamic therapy agent, a progesterone, a radical scavenger, a refatting agent, a retinoid, a sanative, a scabicide, a sedative, a self tanning agent, a skin protective agent, a skin whitening agent, a steroid, a steroid hormone, a vasoactive agent, a vasoconstrictor, a vasodilator, a vitamin, a vitamin A, a vitamin A derivative, a vitamin B, a vitamin B derivative, a vitamin C, a vitamin C derivative, a vitamin D, a vitamin D derivative, a vitamin D analog, a vitamin F, a vitamin F derivative, a vitamin K, a vitamin K derivative, a wound healing agent and a wart remover.

Another aspect of the present invention is directed to methods of treating a skin disorder in patients in need thereof, where the skin disorder is selected from perioral dermatitis, acne vulgaris, rosacea, atopic dermatitis, seborrheic dermatitis, acneiform rashes, transient acantholytic dermatosis, skin wrinkles, facial mottle, hyperpigmentation, hypopigmentation, photo aging, papule, pustule, psoriasis, and lentigo. In some embodiments, the skin disorder is perioral dermatitis.

In certain embodiments, the formulation is applied and rinsed off within from about 0 seconds to about 240 minutes. In other embodiments, the Formulation is applied and rinsed off within about 30 minutes or within about 180 minutes. In certain embodiments, the treatment regimen may continue from about 2 weeks to about 180 days.

Another aspect of the present invention is directed to methods of preparing a stable composition that contains the active ingredients during the shelf life of the product for at least 180 days.

### BRIEF DESCRIPTION OF THE DRAWINGS

The patent or application file contains at least one photograph executed in color. Copies of this patent or patent application publication with color photograph(s) will be provided by the Office upon request and payment of the necessary fee.

FIGS. 1A, 1B, 1C and to 1D (collectively FIG. 1) are photographic images of a subject suffering from moderate to severe erythematous papules and pustules on the face; with FIG. 1A showing a front view and FIGS. 1B and 1D showing side views of the subject's face, and FIG. 1C showing a front view of the subject's neck, with the subject's head directed upward.

FIGS. 2A, 2B to 2C (collectively FIG. 2) are photographic images of a subject who has been treated with the pharmaceutical formulation for moderate to severe erythematous papules and pustules on the face.

### DETAILED DESCRIPTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

The term “pharmaceutical formulation” or “pharmaceutically acceptable formulation: means a formulation which comprises compounds compatible with application to the skin, the mucous membranes and/or the appendages.

The phrase “pharmaceutically acceptable salt(s)”, as used herein, means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the present invention can form pharmaceutically acceptable salts with various amino acids. Suitable base salts include, but are not limited to, aluminum, calcium, lithium, magnesium, potassium, sodium, zinc, and diethanolamine salts.

The term “co-surfactant” as used herein, means a compound which on its own is not able to form and stabilize satisfactorily an oil in water emulsion, but when used in combination with a surfactant, such co-surfactant can boost the emulsifying power of surfactants to create a stable emulsion. For example, fatty alcohols, such as cetyl alcohol or a fatty acid such as stearic acid can function as co-surfactants. Cetyl alcohol and stearyl alcohol are waxy hydrophobic substances that can be emulsified with water using a surfactant. In certain circumstances, a co-surfactant can itself be converted into a surfactant or soap by, for example, adding a base, such as, triethanolamine to a fatty acid, resulting in a fatty acid salt, which is also termed “soap” (a strong anionic surfactant).

The term “hydrate” means a compound of interest, or a pharmaceutically acceptable salt thereof, that further includes a stoichiometric or non-stoichiometric amount of water bound to it by non-covalent intermolecular forces.

The term “polyol”, as used herein, is an organic substance that contains at least two hydroxy groups in its molecular structure.

The term “topical formulation,” as used herein, means any formulation which is pharmaceutically and/or cosmetically acceptable for topical delivery of the specified compounds according to the invention. Exemplary forms of formulation that can be used for topical administration include, but are not limited to, sprays, mists, aerosols, solutions, lotions, gels, creams, ointments, pastes, unguents, emulsions, and suspensions. The choice of topically administrable formulation will depend on several factors, including the nature of the symptoms to be treated or prevented, the physiochemical characteristics of the particular compound to be administered and of other excipients present, their stability in the formulation, available manufacturing equipment, and cost constraints.

As used herein, the term “subject” means a mammal, most preferably a human, to whom will be or has been administered the topical formulations according to embodiments of the invention. The term “mammal” as used herein, encompasses any mammal. Examples of mammals include, but are not limited to, cows, horses, sheep, pigs, cats, dogs, mice, rats, rabbits, guinea pigs, monkeys, humans etc., more preferably, a human. Preferably, a subject is in need of, or has been the object of observation or experiment of treatment or prevention of a skin disorder and symptoms associated therewith.

The term “treatment” or “treating” refers to an amelioration, prophylaxis, or reversal of a disease or disorder, or at least one discernible symptom thereof, for example, treating a skin disorder (e.g., rosacea, perioral dermatitis) by lessening the symptoms such as redness of the skin or improving the skin lesions.

The phrase a “therapeutically effective amount” means the amount of the active ingredient that elicits the biological or medicinal response in a tissue system, animal or human that is being sought by clinical or medical doctor, which includes alleviation of the symptoms of the disease or disorder being treated.

Active Ingredients and Combinations

The formulations of the present invention are directed to a unique combination of active ingredients including an avermectin compound or a pharmaceutically acceptable salt or hydrates thereof; a nitroimidazole compound or a pharmaceutically acceptable salt or hydrates thereof, and a macrolide antibiotics compound or an acceptable salt or hydrate thereof. These formulations are suitable for topical administration to the skin and all areas of the face, including the area surrounding the lips, as well as all mucosal membranes such as nasal cavity, the ear canal and the vaginal cavity. At least one feature of a product for medical use is its prolonged stability of the triple active ingredient and its unique synergistic effect in treating perioral lesions.

In one or more other embodiments, the present formulation contains: a) an avermectin compound or a pharmaceutically acceptable salt or hydrate thereof; b) a nitroimidazole compound or a pharmaceutically acceptable salt or hydrate thereof, and c) a macrolide antibiotics compound or a pharmaceutically acceptable salt or hydrate thereof, and a dermatologically acceptable carrier. In one suitable embodiment, the avermectin compound is selected from ivermectin, invermectin, avermectin, abamectin, doramectin, eprinomectin, selamectin, and any pharmaceutically acceptable salt or ester thereof, in any combinations thereof. In some embodiments, the macrolide compound is selected from erythromycin, clarithromycin, azithromycin, spiramycin, fidaxomicin or any pharmaceutically acceptable salt hydrate or ester thereof in an combinations thereof. In certain embodiments, the nitroimidazole compound is metronidazole, or a pharmaceutically acceptable salt hydrate or ester thereof. In some embodiments the formulation contains ivermectin, ertythromycin and metronidazole moeities.

In the formulations according to the invention, the ivermectin may be present in an amount of from 0.001% to 35%, or from 0.005% to 25% by weight relative to the total weight of the formulation, and preferably from 0.01% to 15%. In certain embodiments, in the formulations according to the invention, the erythromycin is present in an amount of from 0.1% to 30%, from 0.5% to 25% by weight relative to the total weight of the formulation, and or preferably from 0.1% to 15%. In certain embodiments, the metronidazole in the present formulation is present in an amount of from 0.1% to 30% by weight relative to the total weight of the formulation, and preferably from 0.5% to 20%.

In certain embodiments, the formulation of the present invention can further contain one or more additional active agent selected form the group of an active herbal extract, an acaricides, an age spot and keratose removing agent, an allergen, an alpha hydroxyl acid, an analgesic agent, an androgen, an antiacne agent, an antiallergic agent, an antiaging agent, an antibacterial agent, an antibiotic such as tetracycline, an antiburn agent, an anticancer agent, an antidandruff agent, an antidepressant, an antidermatitis agent, an antiedemic anent, an antifungal agent, an antihistamine, an antihelminth agent, an anti-hyperkeratosis agent, an anti-infective agent, an antiinflarnmatory agent, an antiirritant, an antilipemic agent, an antimicrobial agent, an antimycotic agent, an antioxidant, an antiparasitic agent, an antiproliferative agent, an antipruritic agent, an antipsoriatic agent, an antirosacea agent, an antiseborrheic agent; an antiseptic agent, an antiswelling agent, an antiviral agent, an anti-wart agent, an anti-wrinkle agent, an antiyeast agents, an astringent, a beta-hydroxy acid, benzoyl peroxide, a topical cardiovascular agent, a chemotherapeutic agent, a corticosteroid, an immunogenic substance, a dicarboxylic acid, a disinfectant, an estrogen, a fungicide, a hair growth regulator, a haptene, a hormone, a hydroxy acid, an immunosuppressant, an immunoregulating agent, an immunomodula or, an immunostimulant, an insecticide, an insect repellent, a keratolytic agent, a lactam, a local anesthetic agent, a lubricating agent, a masking agent, a metals, a metal oxide, a mitocide, a neuropeptide, a non-steroidal anti-inflammatory agent, an oxidizing agent, a pediculicide, a peptide, a pesticide, a protein, a photodynamic therapy agent, a progesterone, a radical scavenger, a refatting agent, a retinoid, a sanative, a scabicide, a sedative, a self tanning agent, a skin protective agent, a skin whitening agent, a steroid, a steroid hormone, a vasoactive agent, a vasoconstrictor, a vasodilator, a vitamin, a vitamin A, a vitamin A derivative, a vitamin B, a vitamin B derivative, a vitamin C, a vitamin C derivative, a vitamin D, a vitamin D derivative, a vitamin D analog, a vitamin F, a vitamin F derivative, a vitamin K, a vitamin K derivative, a wound healing agent and a wart remover.

### Solvent Systems

The present invention contains at least one hydrophilic, a hydrophobic solvent, a co-solvent, or a combination and mixtures thereof, which provides a skin soothing effect. The selected solvents allow the inclusion of oil-soluble and water-soluble active agents in the formulation. In one or more embodiments, the solvents provide synergistic benefits in combination with the active agent. The compositions may contain at least one oil soluble active agent one or more solvent(s).

Among acceptable solvents that may be employed are water, Ringer's solution and isotonic sodium chloride solution. In addition, sterile, fixed oils are conventionally employed as a solvent or suspending medium, co-solvent, and mixtures thereof, wherein the solvent is present at a concentration of about 35% to about 95% by weight of the total composition.

Choices of other solvents include protic and aprotic solvents and mixtures thereof. For example protic solvents such as short chain alcohols, glycols and glycerin may be compatible with certain actives while incompatible with others. While aprotic polar solvents may be compatible with other active ingredients. To that end, a combination of protic or aprotic may be a suitable system to deliver the combinations of the present invention. Protic polar solvents include dimethyl sulfoxide (DMSO), dimethylformamide (DMF), acetonitrile, acetone, methyl ethyl ketone, 1,4-Dioxane and tetrahydrofuran (THF), N-methylpyrrolidone, pyridine, piperidine, dimethylformanide, N-methyl-2-pyrrolidone and 1-methyl-2-pyrrolidinone) and atone (1-dodecylazacycloheptan-2-one) are undesirable.

Other solvents include propylene glycol, ethanol, isopropanol, butanol, N-methyl-2-pyrrolidone, dimethylsulfoxyde, polysorbate 80, poloxamer 124, phenoxyethanol, oleyl alcohol, isostearic acid, diisopropyl adipate, polypropylene glycol-15 stearyl ether (“PPG-15 stearyl ether”), octyl dodecanol, ethyl oleate, C12-C15 alkyl benzoate and mixtures thereof.

Some hydrophobic solvents are provided to facilitate or enhance the intradermal penetration or delivery of a drug. In one or more additional cases, the hydrophobic solvents are provided to have an occlusive effect at the target site, for example where the site of treatment is a damaged skin and the occlusive effect of hydrophobic solvents is desirable. In certain embodiments, the present application may further include hydrophobic solvents to provide a suitable delivery dosage form. These formulations include semi solid gel formulations that liquefy on application of mild shear force such as gentle rubbing.

In certain dosage forms such as topical foams the formulation may contain higher amounts of water in ranges of about 50% to about 99% by weight of the formulation. In one or more embodiments there is also provided a topical therapeutic hydrophobic breakable formulation containing about 35% to about 99% by weight of at least one hydrophobic oil.

In one or more embodiments, the at least one hydrophobic oil is selected from the group of a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, liquid paraffin, an isoparaffin, a polyalphaolefin, a polyolefin, polyisobutylene, a synthetic isoalkane, isoliexadecane, isododecane, alkyl benzoate, alkyl octanoate, C12-C15 alkyl benzoate, C12-C15 alkyl octanoate, arachidyl behenate, arachidyl propionate, benzyl laurate, benzyl myristate, benzyl palmitate, bis (octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethythexanoate, diethyleneglycol dioctanoate, diethyleneglycol diisononanoate, diethyleneglycol disononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl enicate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, avocado oil, apricot stone oil, barley oil, borage seed oil, calendula oil, canelle nut tree oil, canola oil, caprylicicapric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, rhea butter, soya oil, soybean oil, sweet almond oil, sunflower oil, sysymbrium oil, syzigium aromaticum oil, tea tree oil, walnut oil, wheat germ glycerides, wheat germ oil, PPG-2 butyl ether, PPG-4 butyl ether, PPG-5 butyl, ether, PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, herring oil, cod-liver oil, salmon oil, cyclomethicone, a dimethyl polysiloxane, dimethicone, an epoxy-modified silicone oil, a fatty acid-modified silicone oil, a fluoro group-modified silicone oil, a methylphenylpolysiloxane phenyl trimethicone and a polyether group-modified silicone oil.

In one or more embodiments, the fatty alcohol has at least 12 carbon atoms in its carbon backbone; where the fatty acid has at least 12 carbon atoms in its carbon backbone. In one or more embodiments, the fatty alcohol and the fatty acid have a melting point of more than about 40° C. In one or more embodiments, the fatty alcohol is selected from the group of lauryl alcohol, myristyl alcohol, cetyl alcohol, stearyl alcohol, arachidyl alcohol, behenyl alcohol, tetracosanol, hexacosanol, octacosanol, triacontanol, tetratriacontanol; and wherein said fatty acid is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic, acid, triacontanoic acid, dotriacontanoic acid, tritriacontanoic acid, tetratriacontanoic acid, pentatriacontanoic acid, or any combination thereof.

In one or more embodiments, the carbon chain of the fatty alcohol or the fatty acid is substituted with a hydroxyl group, and for example, the carbon chain of the fatty acid is 12-hydroxy stearic acid.

In some embodiments, the formulation contains an aqueous phase in an amount of from 20% to 95% by weight relative to the total weight of the formulation. In other embodiments, the solvent system may contain a fatty phase having at least one fatty or oily phase which is a solvent for the active agent. The oily phase which is a solvent for the active agent preferably contains at least one oily solvent such as mineral oil or a synthetic oil. Active agents such as those in the avermectin family, preferably ivermectin are particularly soluble in such oil phase. In some embodiments, ivermectin is solubilized in synthetic oils to have improved stability.

In the formulations according to the invention, the aqueous phase is present in an amount of from 1% to 50% by weight relative to the total weight of the formulation.

### Surfactants

Surfactants have been categorized into various classes depending on their ionic characteristics, namely non-ionic surfactants, anionic, cationic, zwitterionic, amphoteric and amphiphilic surfactants. Surfactants of all kinds may be employed in accordance with the present invention, so long as they do not cause degradation of the active ingredients or do not cause skin irritation.

Non-limiting examples of classes of non-ionic surfactants that are undesirable according to the present invention include: (i) polyoxyethylene sorbitan esters (polysorbates), such as polysorbate 20, polysorbate 40, polysorbate 60 and polysorbate 80; (ii) sorbitan esters, such as Sorbitan inonolaurate and sorbitan monooleate; (iii) polyoxyethylene fatty acid esters, such as, PEG-8 Stearate, PEG-20 Stearate, PEG-40 Stearate, PEG-100 Stearate, PEG-150 Distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) polyethylene glycol alkyl phenols.

For certain delivery systems such as topical foam, hydrophobic solvents can have a de-foaming effect which makes the formulation of foams based on hydrophobic solvents challenging. To overcome this challenge, surfactants can provide foaming capabilities and act as foaming agents. The prior art further teaches the incorporation of foam adjuvants, such as fatty alcohols and fatty acids, as foam boosting agents and also the incorporation of polymeric agents (e.g. gelling agents) as foam stabilizers, which can prolong the collapse time of a foam and in the meantime mitigate the irritational properties of the surfactants. Waxes may also be introduced into these surfactant based formulations but as will be appreciated, waxes, which are solids at ambient temperature, can easily precipitate.

In certain embodiments, surface active agents may be eliminated and replaced by viscosity-modifying agents consisting of a fatty alcohol, a fatty acid and a wax in the context of hydrophobic solvent. Waxes are advantageous for providing excellent skin compatibility, almost no chemical reactivity which ensures active ingredients stability, and efficient skin occlusion which helps with reducing skin water loss and can enhance skin penetration of active agents. In alternative embodiments, the term “substantially surfactant-free” relates to a formulation wherein the ratio between the viscosity-modifying agent and the surfactant is between 10:1 or 5:1; or between 20:1 and 10:1 or between 100:1 and 20:1. In such embodiments, the formulations of the present invention may be formulated substantially free of surfactants and or short chain alcohols and or polyols. In one or more other specific embodiments the drug carrier is formulated essentially free of surfactants and/or short chain alcohols and/or polyols. In one or more embodiments there is provided a formulation which is essentially waterless. In one or more embodiments, formulations of the present invention are surfactant-free which may also free of short chain alcohols and or polyol-free.

In some embodiments, the formulation may include an oily phase, an aqueous phase, and a surface active agent. In some embodiments, the formulations according to the invention contain: 0.01% to 25% of oil phase which is a solvent for the active agent; 0.1% to 35% of aqueous phase; and about 0.5% to 25% of surface active agent.

The formulations herein are surprisingly unique in addressing the symptoms of perioral dermatitis. To that end, at least one aspect of the present invention is directed to formulations that provide significantly better clinical outcomes when administered to the perioral regions.

In certain embodiment, the formulations of the present invention surprisingly maintain their potency during the course of treatment and for at least 2 years after exposure to the air. Following accelerated stability studies, they demonstrate desirable texture, do not break immediately upon contact with a surface, spread easily on the treated area and absorb quickly. In one or more embodiments the formulation has an acceptable shelf-life of at least six months. In one or more embodiments the foam formulation has an acceptable shelf-life of at least one year. In one or more embodiments the foam formulation has an acceptable shelf-life of at least 15 months, or at least 18 months, or at least 21 months, or at least two years, at ambient temperature.

### Gelling Agents

As gelling agents that can be used, exemplary are the carbomers marketed under the trademark Carbopol 980 NF and Carbopol 981 NF by Noveon, the C10-C30 alkyl acrylate crosspolymer marketed under the trademark Pemulen TR1 by Noveon, the acrylamide gel marketed under the trademark Simulgel 600 by Seppic, the modified celluloses marketed under the trademark Natrosol by Hercules-Aqualon or Methocel by Dow Chemical Company, or else the saccharide biopolymers marketed under the trademark Xantural by SPCI. In certain embodiments, the formulation contains, 0 to 5% of a gelling agent.

### Thickening Agents

Thickening agents can comprise various types of excipients including silicone thickening agent which can include one or more polysiloxane-derived components. Such polysiloxanes are typically cross-linked and they have rubber-like characteristics, which require their solubilization in an oil, usually a silicone oil. An example of such a silicone thickening agent is ST-Elastomer 10 (Dow Corning), which is a mixture of high molecular weight dimethicone crosspolymer (12%), in cyclopentasiloxane (cyciomethicone, silicone solvent). With reference to bioavailability of an active agent in the skin following topical application, it is conceivable that cross co-polymers will create a non permeable film which should block skin penetration and therefore, it is undesirable. Further, in the context of a breakable foam, cyclomethicone is known as a defoamer and therefore its presence in high concentrations in the breakable hydrophobic Formulation is undesirable.

### Other Excepients

The formulations according to the invention may also contain additives normally employed in the cosmetics or topical pharmaceutical products, such as: humectants such as glycerol, sorbitol or propylene glycol; preservatives such as methyl para-hydroxybenzoate, propyl para-hydroxybenzoate, butyl para-hydroxybenzoate, phenoxyethanol, benzalkonium chloride, benzyl alcohol, phenylethyl alcohol, chlorhexidine digluconate or chlorephenesin; anti-irritants, such as allantoin, 18.beta.-glycyrrhetinic acid or DL-alpha-tocopheryl acetate; moisture regulators; pH regulators, such as citric acid or sodium hydroxide; osmotic pressure modifiers; UV-A and UV-B screens; and antioxidants, such as .alpha.-tocopherol, butylhydroxyanisole (BHA) or butylhydroxytoluene (BHT), vitamin E, propyl gallate or citric acid.

One skilled in this art will adapt the choice of the additives or the optional compounds to be added to these formulations and also the procedure in such a way that the advantageous properties intrinsically associated with the present invention are not, or are not substantially impaired by the addition envisaged.

For example, in one or more embodiments the viscosity of the combination may be higher than about 10000 cPs; or between about 1000 cPs and about 100000 cPs; or between about 5000 cPs and about 50000 cPs; or between about 10000 cPs and about 30000 cPs. To that end, these additives may be present in the formulation at from 0.001% to 25% by weight relative to the total weight of the formulation to provide the physical characteristics that achieve the best clinical outcome. In other embodiments, the formulation is mixed in a manner to create an emulsion having a hydrophilic/lipophilic balance (HLB) of more than about 10.

### Foaming Propellant

In certain embodiments, the present formulation may be in the form of a foam requiring a suitable propellant, which can include compressed gases, volatile hydrocarbons such as butane, propane, isobutane and fluorocarbon gases, or mixtures thereof. A foamable Formulation manufactured according to one or more embodiments herein is very easy to use. When applied onto the afflicted body surface of mammals, i.e., humans or animals, it is in a foam state, allowing free application without spillage. Upon further application of a mechanical force, e.g., by rubbing the Formulation onto the body surface, it freely spreads on the surface and is rapidly absorbed.

### Formulations

In at least one example of the present formulations the ratio of ivermectin to metronidazole to erythromycin their acceptable salts or hydrates thereof is in the ranges of 0.5%-7.5% per weight, 1% to 5% per weight and 3% to 10% per weight of the formulation respectively. In some embodiments, the ivermactin amount ranges between 1% to 4% per weight, while the amount of metronidazole ranges from about 1.5% to 4.0% per weight, and erythromycin ranges from about 4% to about 7.5% per weight of the entire formulation.

In one embodiment, ivermectin at 1%, metronidazole at 0.75%, and erythromycin at 2% base were mixed together at a 1:1:1 ratio to formulate a homogenous combination. In certain embodiment, the present invention may contain a combination of 1% ivermectin cream base, 0.7% metronidazole ointment and 0.2 aqueous erythromycin base at 2% in the form of a hydrogel of a base.

Ivermectin is a semi-synthetic derivative isolated from the fermentation of Streptomyces avermitilis that belongs to the avermectin family of macrocyclic lactones. Ivermectin is a mixture containing not less than 95.0% and not more than 102.0% of 5-O-demethyl-22,23-dihydroavermectin Ala plus 5-O-demethyl-25-de(1-methylpropyl)-25-(1-methylethyl)-22,23-dihydroavermectin Ala, generally referred to as 22,23-dihydroavermectin B1a and B1b or H2B1a and H2B1b, respectively; and the ratio (calculated by area percentage) of component H2B1a/(H2B1a+H2B1b)) is not less than 90.0%. Ivermectin base cream, 1% is a white to pale yellow hydrophilic cream. Each gram of such cream contains 10 mg of ivermectin.

Metronidazole is 2-methyl-5-nitro-1H-imidazole-1-ethanol. In some embodiments, metronidazole cream base may contain a concentration of 7.5 mg per gram (0.75%) in an emollient cream.

In other embodiments, erythromycin is in the form of an aqueous topical gel base containing 20 mg of erythromycin, USP in a base of alcohol 92% and hydroxypropyl cellulose.

In other embodiments, the present formulation may contain other ingredients such as a surfactant, thickening agent preservative and buffer adjusters including but not limited to carbomer copolymer type B, cetyl alcohol, citric acid monohydrate, dimethicone, edetate disodium, glycerin, isopropyl palmitate, methylparaben, oleyl alcohol, phenoxyethanol, polyoxyl 20 cetostearyl ether, propylene glycol, propylparaben, purified water, sodium hydroxide, sorbitan monostearate, stearyl alcohol, benzyl alcohol, emulsifying wax, glycerin, isopropyl palmitate, purified water, sorbitol solution, lactic acid and/or sodium hydroxide to adjust pH.

### Methods

The present invention also features a method of making and using the present formulations. The methods for preparing the subject formulations include the steps: a) mixing the active ingredients in the suitable solvent system, until all actives are solubilized, b) optionally mixing the constituents of the aqueous phase, to homogeneity; c) incorporating additional excepients into the final mixture.

This invention also features administration of the subject formulations for treating dermatological conditions. These conditions include particularly perioral dermatitis and rosacea. Perioral dermatitis is a condition related to acne vulgaris, and consists of red papules that may appear microvesicular and that typically affect the nasolabial folds (around the nostrils), perioral area (around the mouth) or perioccular area (around the eyes). Rosacea is a chronic condition characterized by facial erythema (redness) and sometimes pimples.

One aspect of the present invention is directed to methods of treating pateints who are suffering from such perioral dermatitis or rosacea by applying and optionally rinsing off the formulation within from about 0 seconds to about 240 minutes, or preferably 15 seconds to about 30 minutes or within about 30 seconds to about 15 minutes. In certain embodiments, the treatment regimen may continue from about 1 week to about 180 days. In certain embodiments, the patient may apply the present combination once a day. In certain embodiments, the patient may apply the present combination twice a day.

The present formulation is also beneficial among patients suffering from conditions such as acne vulgaris, atopic dermatitis, seborrheic dermatitis, acneiform rashes, transient acantholytic dermatosis, skin wrinkles, facial mottle, hyperpigmentation, hypopigmentation, photo aging, papule, pustule, psoriasis, and lentigo.

In at least some embodiments, the method of treatment further includes a step of monitoring the clinical efficacy of the disclosed treatment regimen by clinical outcome measures and indexes customized for individual patients suffering from the above mentioned dermatological conditions. Such outcome measures include: eczema area and severity index (EAST), investigators global assessment scales (IGA), scoring atopic dermatitis (SCORAD), pruritis numerical rating scale (NRS), dermatology life quality index (DLQI), patient-oriented eczema measure (POEM), or other knows indexes for the same purpose.

In preferred embodiments, the evaluation of patients suffering from perioral dermatitis after applying the formulations of the present invention may include using such assessments as perioral dermatis severity index (PODSI). This index allows for precision in results when comparing the placebo population to those that had received the present formulation. The index also allows for simple mathematical modeling since it produces numerical values based on the severity of a patient's condition. While the PODSI is a good index for condition improvement, it does not take into account the patient's input so it should be used in conjunction with other modes of evaluation.

To account for the patient's perspective, the present method of treatment also employs the Dermatology Life Quality Index (DLQI) along with the PODSI. DLQI is a 10-question patient questionnaire that is filled out at every physician visit. The questionnaire covers different aspects that may affect the patient's quality of life while suffering from their condition. The index covers symptoms and feelings, daily activities, leisure, work and school performance, personal relationships, and treatment. In addition, this index has shown statistically significant reliability and responsiveness in several studies, suggesting it would be a favorable index to use in evaluation of the presently disclosed formulations for treatment of perioral dermatits.

Another commonly used Index for evaluation in clinical trials is the Investigator's Global Assessment Scale (IGA). This scale is used frequently in dermatology clinical trials and was designed for that purpose. It is also often used as a basis for FDA approval.

As the present methods of treatment include an intermittent monitoring of the skin conditions by employing at least one of the PODSI, DLQI or IGA during the treatment regimen.

In some embodiments, the present formulation is stored under cool temperatures, which can improve the ease of application. In some cases, the formulation is stored at temperatures of from about 0° C. to about 4° C.

In order to further illustrate the present invention and the advantages thereof, the following specific examples are given, it being understood that same are intended only as illustrative and in no way limitative. In said examples to follow, all parts and percentages are given by weight, unless otherwise indicated.

### EXAMPLES

### Example 1—Formulation

A formulation was prepared by adding 360 grams of metronidazole 0.75%, 360 grams of soolantra (ivermectin 1%) and 360 grams of erythromycin 2%, and mixing well by stirring to form a homogenous combination. The contents of the container were distributed amongst several 2 oz jars.

### Example 2—Treatment

A 26 year old female subject was seen for a chief complaint of “Acne” located on the face. The lesions consisted of papules and pimples, was severe in severity, and had been present for months. Pertinent negatives included: the “acne” did not worsen around the subject's menstrual cycle, the subject did not have a face that was oily or dry, the subject did not not pick at the “acne”, the subject did not wear make up, and did not have a history of accutane use, oral contraceptive use, or radiation treatment for “acne.” The subject's last menstrual period was 10/05/2020. The subject had previously tried Doxycycline and tretinoin, with no improvement in the past. The subject was having a bad “acne” flare up, and stated that her face was itchy and red. The subject had been treated for “acne” for a long time.

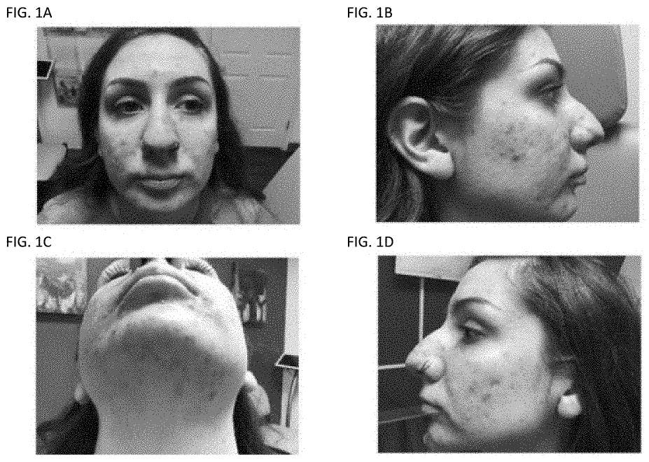

Upon examination, the subject was found to have erythematous papules and pustules on the cheeks forehead, chin, around nose, and periorally. The subject's condition at examination was recorded in the images shown in FIG. 1A to 1D. The subject was diagnosed with moderate to severe papular pustular rosacea with perioral dermatitis.

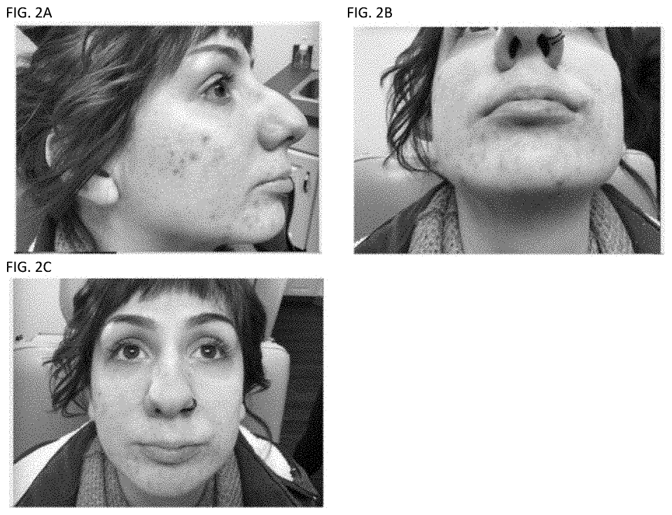

The subject was given the formulation of Example 1, and the subject administered it twice daily (AM and PM) and was seen again 1 month later, at which time her condition had improved. The subject's condition at this later examination was recorded in the images in FIGS. 2A to 2C.

What is claimed is:

1. A Formulation comprising: a) an avermectin compound or a pharmaceutically acceptable salt thereof; b) a nitroimidazole compound or a pharmaceutically acceptable salt thereof, and c) a macrolide antibiotics compound or a pharmaceutically acceptable salt thereof, and a dermatologically acceptable carrier.

2. The Formulation of claim 1, wherein the carrier comprises a surfactant that is selected from the group consisting of anionic, cationic, nonionic, amphoteric surfactants and a mixture thereof

3. The Formulation of claim 1, wherein the carrier comprises a fatty substances selected from the group consisting of fatty acids, fatty alcohols, waxes, gums, and mixtures thereof.

4. The Formulation of claim 1, wherein the carrier comprises 0.1 to 50% by weight of solvents.

5. The Formulation of claim 4, wherein the solvents are selected from the group consisting of polar, non-polar and a mixture of polar and non-polar solvents.

6. The Formulation of claim 5, wherein the solvents are selected from the group consisting of propylene glycol, ethanol, isopropanol, butanol, N-methyl-2-pyrrolidone, dimethylsulfoxyde, polysorbate 80, poloxamer 124, phenoxyethanol, oleyl alcohol, isostearic acid, diisopropyl adipate, polypropylene glycol-15 stearyl ether (“PPG-15 stearyl ether”), octyl dodecanol, ethyl oleate, C12-C15 alkyl benzoate and mixtures thereof.

7. The Formulation of claim 1, wherein the avermectin compound is selected from the group consisting of ivermectin, invermectin, avermectin, abamectin, doramectin, eprinomectin, selamectin and a combination thereof.

8. The Formulation of claim 7, wherein the avermectin compound is ivermectin.

9. The Formulation of claim 7, wherein the avermectin compound is present in a concentration ranging from 0.01 to 30% by weight, relative to the total weight of the Formulation.

10. The Formulation of claim 9, wherein the avermectin compound is present in a concentration ranging from 0.1 to 5% by weight, relative to the total weight of the Formulation.

11. The Formulation of claim 1, wherein the macrolide compound is selected from the group consisting of erythromycin, clarithromycin, azithromycin, spiramycin, fidaxomicin and any combinations thereof.

12. The Formulation of claim 11, wherein the macrolide compound is erythromycin.

13. The Formulation of claim 1, wherein the nitroimidazole compound is metronidazole.

14. The Formulation of claim 2, wherein the surfactant is a non-ionic surfactant selected from the group consisting of polyoxyethylenated fatty alcohol ethers, sorbitan esters, and mixtures thereof.

15. The Formulation of claim 1, wherein and the Formulation has a viscosity within a range of from about 1,000 centipoise to about 100,000 centipoise at 25° C.

16. The Formulation of claim 1, further comprising an additional active ingredient selected from the group consisting of a retinoid, tetracycline, vitamin E, zinc salt, and skin penetrating enhancer, and a combination thereof.

17. The Formulation according to claim 1, having a hydrophilic/lipophilic balance (HLB) of more than about 10.

18. A method of treating a skin disorder comprising administering the Formulation of claim 1 to a patient in need thereof, wherein the skin disorder is selected from the group consisting of perioral dermatitis, acne vulgaris, rosacea, atopic dermatitis, seborrheic dermatitis, acneiform rashes, transient acantholytic dermatosis, skin wrinkles, facial mottle, hyperpigmentation, hypopigmentation, photo aging, papule, pustule, psoriasis, and lentigo.

19. The method of claim 18, wherein the Formulation is applied and rinsed off within from about 0 seconds to about 240 minutes.

20. The method of claim 19, wherein the Formulation is applied and rinsed off within about 30 minutes or within about 180 minutes.

21. The method of claim 18, wherein the skin disorder is peri-oral dermatitis.

22. The method of claim 18, wherein the skin disorder is rosacea.
